(19) United States
(12) Patent Application Publication
(10) Pub. No.: US 2024/0296952 A1
(43) Pub. Date: Sep. 5, 2024

(54) SYSTEMS AND METHODS FOR DEEP LEARNING BASED ELECTROCARDIOGRAPHIC SCREENING FOR CHRONIC KIDNEY DISEASE

(71) Applicant: Cedars-Sinai Medical Center, Los Angeles, CA (US)

(72) Inventors: David Ouyang, Los Angeles, CA (US); Lauri Holmstrom, Los Angeles, CA (US); Matthew Kai Christensen, Los Angeles, CA (US)

(73) Assignee: Cedars-Sinai Medical Center, Los Angeles, CA (US)

(21) Appl. No.: 18/595,237

(22) Filed: Mar. 4, 2024

Related U.S. Application Data
(60) Provisional application No. 63/488,334, filed on Mar. 3, 2023.

(51) Int. Cl. G16H 50/20; A61B 5/0245; A61B 5/353; A61B 5/355; A61B 5/36; A61B 5/366; G16H 10/60; G16H 50/30

(52) U.S. Cl. CPC G16H 50/20; A61B 5/0245; A61B 5/353; A61B 5/355; A61B 5/36; A61B 5/366; G16H 10/60; G16H 50/30

(57) ABSTRACT

A method for analyzing kidney health in an individual comprises receiving data associated with one or more cardiac characteristics of the individual; inputting the data associated with the one or more cardiac characteristics of the individual into a machine learning model; and receiving an output from the machine learning model indicative of the kidney health of the individual. The output of the machine learning model can include an indication of the presence of chronic kidney disease (CKD) in the individual or an indication of the absence of CKD in the individual. The indication of the presence of CKD in the individual can include an indication of the CKD stage of the individual, which may include mild CKD, moderate-severe CKD, or end-stage renal disease ESRD).

### CROSS-REFERENCE TO RELATED APPLICATIONS

This application claims the benefit of, and priority to, U.S. Provisional Patent Application No. 63/488,334 filed on Mar. 3, 2023, which is hereby incorporated by reference herein in its entirety.

### STATEMENT REGARDING FEDERALLY SPONSORED RESEARCH

This invention was made with government support under Grant No. HL157421 awarded by the National Institutes of Health. The government has certain rights in the invention.

### TECHNICAL FIELD

The present disclosure relates generally to systems and methods analyzing kidney health in an individual, and more particularly, to systems and methods for performing deep-learning analysis of electrocardiographic data to screen for chronic kidney disease.

### BACKGROUND

Undiagnosed chronic kidney disease (CKD) is a common and usually asymptomatic disorder that causes a high burden of morbidity and early mortality worldwide. New techniques for CKD screening are needed.

### SUMMARY

According to some implementations of the present disclosure, a method for analyzing kidney health in an individual is disclosed. The method includes receiving data associated with one or more cardiac characteristics of the individual. The method further includes inputting the data associated with the one or more cardiac characteristics of the individual into a machine learning model. The method further includes receiving an output from the machine learning model indicative of the kidney health of the individual. In some implementations, the output of the machine learning model includes an indication of a presence of chronic kidney disease (CKD) in the individual or an indication of an absence of CKD in the individual. In some implementations, the indication of the presence of CKD in the individual includes an indication of a CKD stage of the individual. In some implementations, the CKD stage of the individual includes mild CKD, moderate-severe CKD, or end-stage renal disease (ESRD). In some implementations, the data associated with the one or more cardiac characteristics of the individual includes 12-lead ECG data, 1-lead ECG data, or both. In some implementations, the one or more cardiac characteristics of the individual includes a heart rate, a PR interval, a P wave duration, a QRS duration, a QTc interval, a P-wave axis, an R-wave axis, a T-wave axis, or any combination thereof. In some implementations, the machine learning model includes a convolutional neural network trained to identify a presence of CKD in the individual, to determine a stage of the CKD in the individual, or both.

According to some implementations of the present disclosure, a system for analyzing kidney health of an individual is disclosed. The system includes at least one electronic interface, at least one memory device, and at least one processing device. The at least one electronic interface is configured to receive data associated with one or more cardiac characteristics of the individual. The at least one memory device is configured to store the data associated with the one or more cardiac characteristics of the individual. The at least one processing device configured to implement a machine learning model, the machine learning model being configured to receive the data associated with the one or more cardiac characteristics of the individual and output an indication of the kidney health of the individual

The above summary is not intended to represent each implementation or every aspect of the present disclosure. Additional features and benefits of the present disclosure are apparent from the detailed description and figures set forth below.

### BACKGROUND

The foregoing and other advantages of the present disclosure will become apparent upon reading the following detailed description and upon reference to the drawings.

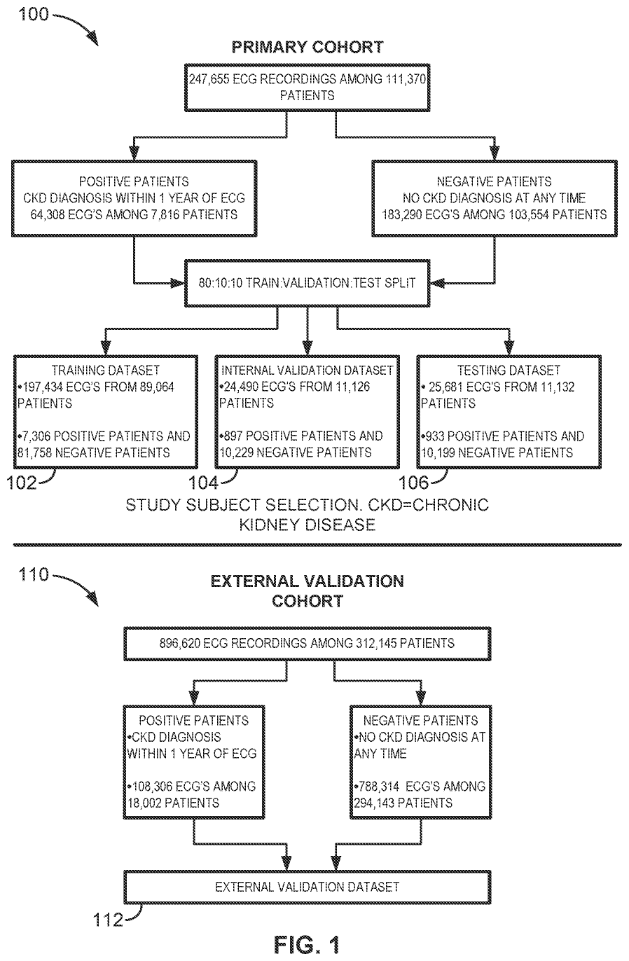

FIG. 1 shows the primary cohort used to form a internal training cohort, an internal validation cohort, and an internal testing cohort for one or more machine learning models to analyze kidney health, and the external validation cohort used to further validate the machine learning models, according to aspects of the present disclosure.

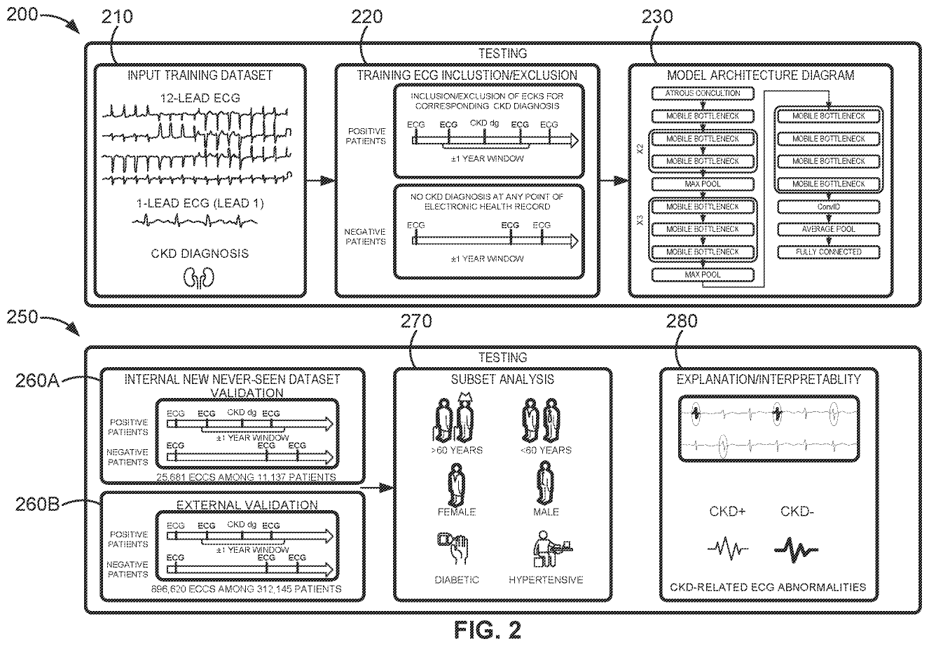

FIG. 2 shows flowcharts of the processes for training and testing the machine learning models, according to aspects of the present disclosure.

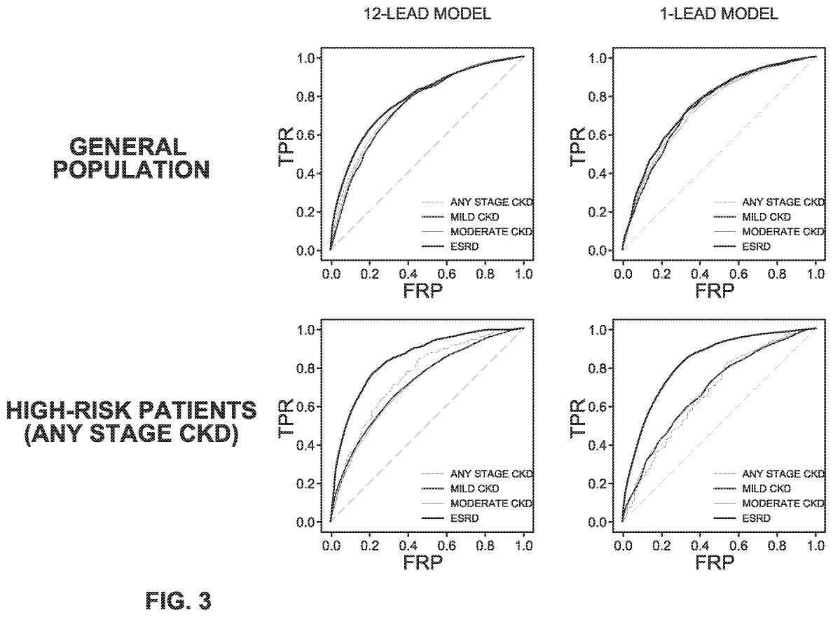

FIG. 3 shows true positive rate vs. false positive rate graphs for the 12-lead ECG models and the 1-lead ECG models, according to aspects of the present disclosure.

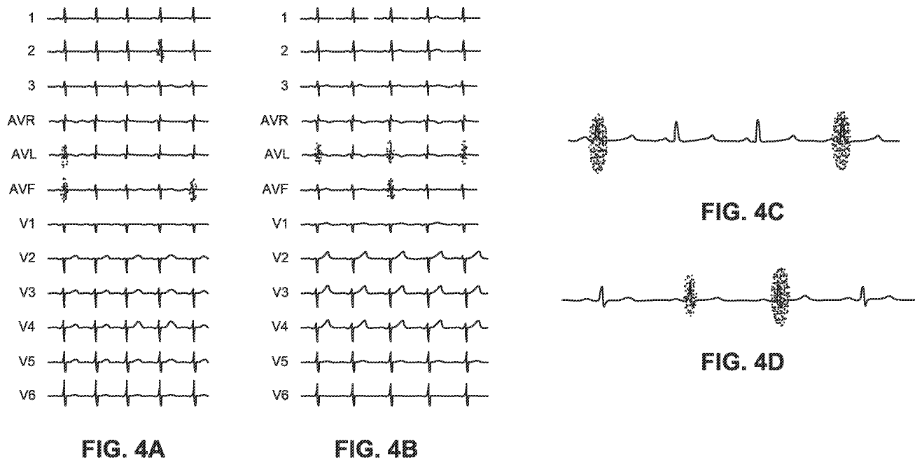

FIG. 4 shows linear interpretable model-agnostic explanation maps for 12-lead and 1-lead ECGs, according to aspects of the present disclosure.

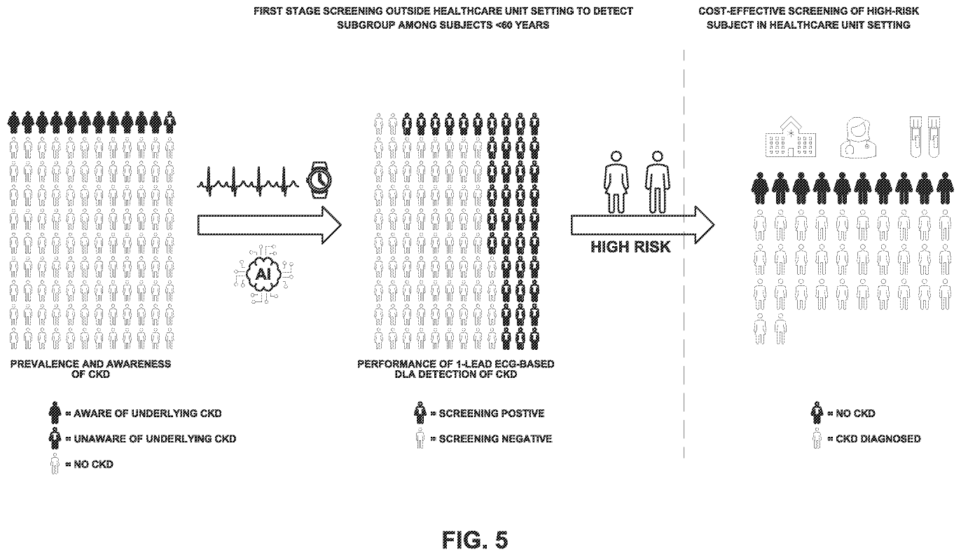

FIG. 5 shows how ECG-based machine learning models can be used to detect a CKD high-risk subgroup, according to aspects of the present disclosure.

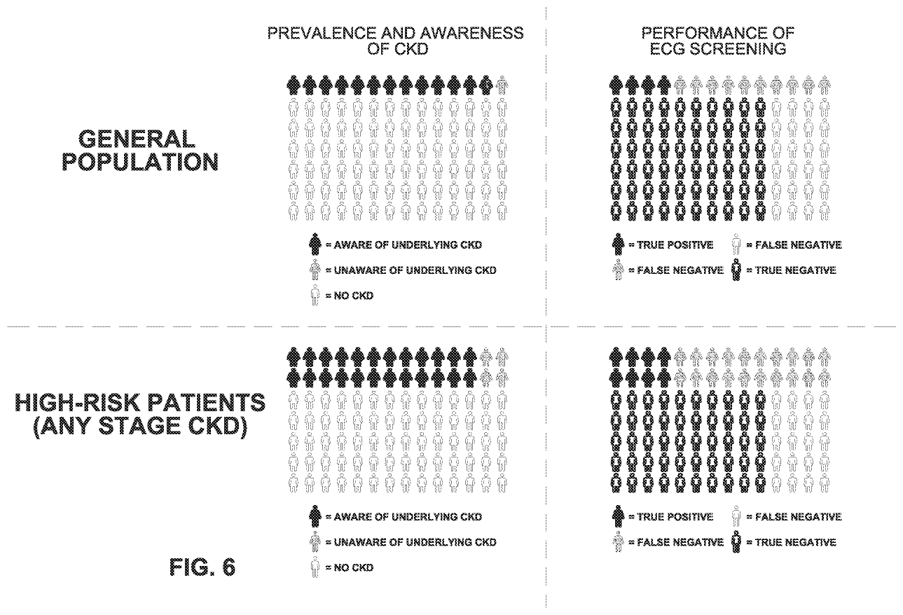

FIG. 6 shows CKD prevalence and awareness compared to the performance of the one or more machine learning models, according to aspects of the present disclosure.

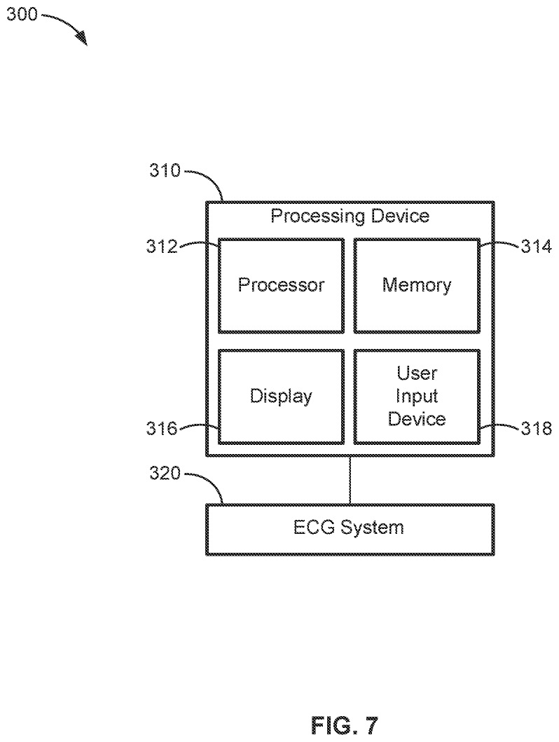

FIG. 7 is a block diagram of a system for analyzing the health of a kidney of an individual, according to aspects of the present disclosure.

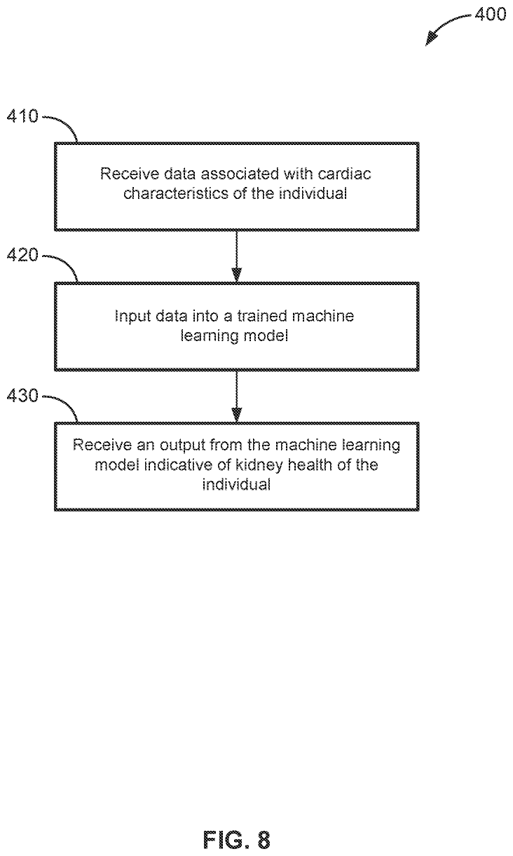

FIG. 8 is a flow chart of a method for analyzing the health of a kidney of an individual, according to aspects of the present disclosure.

While the present disclosure is susceptible to various modifications and alternative forms, specific implementations and embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

Disclosed herein are systems and methods for screening for chronic kidney disease (CKD) using one or more machine learning models that analyze electrocardiographic (ECG) data, including 1-lead ECG data and 12-lead ECG data.

Almost 700 million individuals globally have chronic kidney disease (CKD), an important but often unrecognized cause of morbidity and early mortality. The initial presentation of CKD is usually asymptomatic and without overt clinical manifestations especially in early stages of the disease. Recently, the Global Burden of Diseases, Injuries and Risk Factors Study (GBD) estimated that CKD accounts for 4.6% of total mortality worldwide, with a 41.5% increase between 1990-2017. Delayed diagnosis and limited patient recognition of the condition contributes significantly to the burden of morbidity. Early detection can potentially change the disease trajectory. The most common causes of CKD, such as hypertension and diabetes, can be reversible or treatable, and early diagnosis is crucial for avoiding renal replacement therapy. There are few methods to cheaply or non-invasively screen for CKD, with conventional risk calculators lacking specificity and requiring both serum and urine laboratory testing.

Electrocardiograms (ECGs) are inexpensive, non-invasive, widely available, and rapid diagnostic tests frequently obtained during routine visits, prior to exercise, during preoperative evaluation, and for patients at increased risk of cardiovascular disease. Deep learning algorithms (DLA) (also referred to herein as machine learning models) have recently been applied to medical imaging and clinical data to achieve high precision, and to identify additional information beyond the interpretation of human experts. Deep learning analysis of ECG waveforms has had potentially promising performance in prognosticating outcomes, identifying subclinical disease, and identifying systemic phenotypes not traditionally associated with ECGs. Given the prior success in identifying occult arrhythmias, ventricular dysfunction, anemia, and age, DLA applied to screening ECGs could potentially identify patients who would benefit from further evaluation for kidney disease.

The high prevalence of concomitant cardiovascular disease and the well-established changes that accompany electrolyte abnormalities suggest that the ECG is also altered in the setting of CKD and that discrete electrocardiographic signatures could be identifiable with deep learning techniques. Patients with CKD have a disproportionate accumulation of CV risk factors, such as diabetes and hypertension, as well as subclinical cardiovascular changes such as left ventricular hypertrophy, myocardial fibrosis, and diastolic dysfunction. It is not fully clear at which stage CKD patients start to develop manifest cardiovascular changes. However, recent studies have reported that patients with early-stage CKD may already have an increase in diffuse myocardial fibrosis on cardiac MRI. In addition to myocardial remodeling, CKD associates with a variety of electrolyte abnormalities that also cause widespread ECG abnormalities (e.g., decreased T-wave amplitudes in hypokalemia, large-amplitude T-waves, and prolonged QRS duration in hyperkalemia, and QTc prolongation in hypocalcemia. Given such observations, it may be possible that asymptomatic CKD presents with subtle ECG alterations that are not visible to the human eye.

To overcome current limitations in screening for occult CKD, a machine learning/deep learning model was designed, trained, and validated to predict CKD, including end-stage renal disease (ESRD), by analysis of waveform signals from a single 12-lead and 1-lead ECG. Incorporating both structured information from medical diagnoses as well as laboratory data, the ability of the model to evaluate the entire spectrum of kidney disease was assessed. To further evaluate the model, its performance was validated using corresponding data from a separate healthcare system.

54,582 ECGs were retrospectively identified among 7,947 patients between 2005-2019 which were linked to a diagnosis of CKD within a 1-year window at Cedars-Sinai Medical Center. Also identified were 193,073 ECGs among 103,814 patients between 2008-2019 with no CKD diagnoses at any point, which were used as matched negative controls. The study population from CSMC was randomly split 8:1:1 into training, validation, and test cohorts by patient such that the multiple ECGs from the same patient were limited to one cohort. In addition, 896,620 ECGs were identified among 312,145 patients at Stanford Healthcare from August/2005 to June/2018, which were used for external validation. FIG. 1 illustrates the primary cohort 100 used to form the internal training dataset 102, the internal validation dataset 104, and the internal testing dataset 106. FIG. 1 also illustrates the external validation cohort 110 used to form the external validation data set 112, which can be used to validate the disclosed models.

The ECG waveform data were acquired at a sampling rate of 500 Hz and extracted as 10 second, 12×5000 matrices of amplitude values. ECGs with missing leads were excluded from the study cohort. Associated clinical data for each patient was obtained from the electronic health record (EHR). Disease diagnoses were identified by International Classification of Diseases (ICD) 9th edition codes and demographic and clinical characteristics (e.g., age, gender, BMI, cardiovascular disease) were also extracted from the electronic health records.

FIG. 2 shows flowcharts 200 and 250 for training and testing a convolutional neural network designed to predict the primary outcomes of chronic kidney disease and end-stage renal disease based on ECG data. Block 210 of flowchart 200 includes collecting the ECG data from the internal training dataset 102, which can include 12-lead ECG data, 1-lead ECG data, and an indication of whether each patient was diagnosed with CKD. Block 220 includes training the model with ECG data from positive patients (diagnosed with CKD) and ECG data from negative patients (no diagnosis of CKD). At block 230, the final model is full trained. In the disclosed implementation, separate 12-lead ECG and 1-lead ECG models were generated. In other implementations, a model that uses both 12-lead ECG data and 1-lead ECG data can be used.

The model was trained to predict outcomes with the input of one 12-lead ECG obtained within 1 year of diagnosis. If the same patient had multiple ECGs, each was considered an independent case. Models were trained using the PyTorch deep learning framework. The model was initialized with random weights and trained using a binary cross-entropy loss function for up to 100 epochs with an ADAM optimizer and an initial learning rate of 1e-4. Early stopping was performed based on the validation dataset's area under the receiver operating curve.

Block 260A of flowchart 250 includes performing internal validation with the internal validation dataset 104, and block 260B includes performing external validation with the external validation set 112. Block 270 includes performing analysis of different subsets of patients, including patients greater than or equal to 60 years old, patients less than or equal to 60 years old, female patients, male patients, diabetic patients, and hypertensive patients. Other subsets of patients may also be analyzed. Block 280 include performing Local Interpretable Model-agnostic Explanations (LIME) tests to identify relevant features in the ECG waveform by iteratively randomly perturbing 0.5% of the waveform and identifying which changes most impacted model performance.

All analyses were performed on the held-out test dataset, which was never seen during model training. The performance of the model in predicting the primary outcomes was mathematically assessed by the area under the curve (AUC) of the receiver operating characteristic (ROC) curve. After model derivation and training, primary and secondary analyses were performed on trained models using the held-out test cohort. Secondary sensitivity analyses were limited to procedures performed in patients with diabetes, hypertension, male, and age greater or lower than 60 years old. Two-sided 95% confidence intervals were computed using 1,000 bootstrapped samples for each calculation. Statistical analysis was performed in R and Python.

The primary cohort consisted of a total of 247,655 ECGs, of which 221,974 were randomized to the training set (for both training and validation) and 25,681 to the testing set. The number of patients in the training set was 100,233 of which 0.8% had mild CKD, 3.5% had moderate-severe CKD, and 2.7% had ESRD. The testing set included 11,137 patients of which 0.7% had mild CKD, 3.6% had moderate-severe CKD, and 2.8% had ESRD. The mean age of the primary cohort was 61.3±19.7 years and 48% were female. The proportion of Caucasians was 60.4%, whereas 13.8% were black, 5.5% were Asians, and 20.3% had other or unknown race. Demographic and clinical characteristics are presented in Table 1 below. In Table 1, continuous variables are presented as mean t standard deviation, BMI=body mass index, eGFR=estimated glomerular filtration rate, and ESRD=end-stage renal disease. The eGFR data was measured within a month of the ECG data.

TABLE 1

| | | | | | | | External | |
|---|---|---|---|---|---|---|---|---|
| | Internal training and validation datasets | | | | | | validation | |
| Characteristic | Total | | Training | | Test | | dataset | |
| Number of patients | 111,370 | | 100,233 | | 11,137 | | 312,145 | |
| Number of ECGs | 247,655 | | 221,974 | | 25,681 | | 896,620 | |
| Demographics | | | | | | | | |
| Age, years | 61.3 ± 19.7 | | 61.3 ± 19.6 | | 61.5 ± 19.9 | | 56.7 ± 18.7 | |
| Female, n (%) | 53,476 | (48.0%) | | | | | | |
| BMI, kg/m2 | 26.7 ± 16.6 | | 26.7 ± 18.2 | | 26.6 ± 7.2 | | 27.6 ± 6.6 | |
| Caucasian, n (%) | 67,253 | (60.4%) | 60,550 | (60.4%) | 6,703 | (60.2%) | 148,367 | (47.5%) |
| Black, n (%) | 15,323 | (13.8%) | 13,751 | (13.7%) | 1,572 | (14.1%) | 11,191 | (3.6%) |
| Asian, n (%) | 6,135 | (5.5%) | 5,558 | (5.5%) | 577 | (5.2%) | 38,229 | (12.3%) |
| Other/unknown race, n (%) | 22,659 | (20.3%) | 20,374 | (20.3%) | 2,285 | (20.5%) | 114,358 | (36.6%) |
| Clinical Characteristics, n (%) | | | | | | | | |
| Hypertension | 25,446 | (26.4%) | 22,623 | (26.4%) | 2,823 | (26.3%) | 110,311 | (35.3%) |
| Diabetes Mellitus | 8,728 | (7.8%) | 6,970 | (7.8%) | 914 | (8.2%) | 14,152 | (4.5%) |
| Cardiovascular disease | 15,719 | (14.1%) | 12,561 | (14.1%) | 1,577 | (14.2%) | 34,582 | (11.1%) |
| Heart Failure | 9,604 | (8.6%) | 7,634 | (8.6%) | 995 | (8.9%) | 20,167 | (6.5%) |
| Proteinuria | 1,007 | (0.9%) | 795 | (0.9%) | 110 | (1.0%) | 2,086 | (0.7%) |
| Anemia | 11,933 | (10.7%) | 9,537 | (10.7%) | 1,194 | (10.7%) | 18,653 | (6.0%) |
| Chronic Kidney Disease, n (%) | | | | | | | | |
| Mild (Stage 1-2) | 864 | (0.8%) | 782 | (0.8%) | 82 | (0.7%) | 3,727 | (1.2%) |
| Moderate-severe (Stage 3-5) | 3918 | (3.5%) | 3515 | (3.5%) | 403 | (3.6%) | 11,335 | (3.6%) |
| ESRD | 3034 | (2.7%) | 2722 | (2.7%) | 312 | (2.8%) | 2,940 | (0.9%) |
| eGFR, ml/min/1.73 m² | | | | | | | | |
| <15 | 1,744 | (7.0%) | 1,531 | (7.0%) | 213 | (7.7%) | | |
| 15-29 | 1,789 | (7.2%) | 1,563 | (7.1%) | 226 | (2.0%) | | |
| 30-60 | 5,477 | (22.1%) | 4,831 | (22.0%) | 646 | (23.3%) | | |
| >60 | 15,575 | (62.9%) | 13,904 | (63.3%) | 1,671 | (60.3%) | | |
| Potassium (mmol/L) | | | | | | | | |
| >5.5 | 2,356 | (2.7%) | 2,075 | (2.7%) | 281 | (2.9%) | | |
| <=5.5 | 85,573 | (97.3%) | 76,106 | (97.3%) | 9,467 | (97.1%) | | |

The 12-lead ECG-based model achieved discrimination of any stage CKD with an AUC of 0.767 (95% CI 0.76-0.773). The model performance was consistent across the range of CKD stage, with the model achieving an AUC of 0.753 (0.735-0.770) in discriminating mild CKD, AUC of 0.759 (0.750-0.767) in discriminating moderate-severe CKD, and AUC of 0.783 (0.730-0.752) in discriminating ESRD. In all cases, negative examples were defined as ECGs without CKD diagnoses. Sensitivity and specificity at detecting any stage CKD were 0.699 (0.699-0.699) and 0.698 (0.698-0.698), respectively.

Given the increased prevalence of wearable technologies, particularly devices that include single lead ECG information, an additional model was trained with information from only single lead ECG information to simulate the model's performance with single-lead wearable information. With 1-lead ECG waveform data, the model achieved an AUC of 0.744 (0.737-0.751) in detecting any stage CKD, with sensitivity and specificity of 0.723 (0.723-0.723) and 0.643 (0.643-0.643), respectively. In addition, 1-lead ECG-based model achieved an AUC of 0.746 (0.728-0.764) in detecting mild CKD, AUC of 0.735 (0.726-0.744) in detecting moderate-severe CKD, and AUC of 0.757 (0.748-0.767) in detecting ESRD.

Since early detection of CKD is crucial to prevent disease progression and complications in older age, the performance of the was tested model in younger patients (<60 years of age). 12-lead and 1-lead ECG-based models were able to detect any stage CKD with AUCs of 0.843 (0.834-0.852) and 0.824 (0.814-0.833) among patients under 60 years of age, respectively. Sensitivity in detecting any stage CKD was 0.761 (0.761-0.761) with 12-lead ECG waveform and 0.812 (0.812-0.812) with 1-lead ECG waveform. Specificities were 0.787 (0.787-0.787) and 0.705 (0.705-0.705) for 12-lead and 1-lead ECG waveforms, respectively.

The performance of the model was tested separately among diabetic, hypertensive, older patients, who are generally considered as high-risk subgroups. 12-lead based model detected CKD with an AUC of 0.747 (0.707-0.783) among diabetic patients, an AUC of 0.714 (0.701-0.726) among patients with hypertension, and an AUC of 0.706 (0.697-0.716) among patients greater than 60 years old. Similarly, the 1-lead ECG-based model achieved discrimination of any stage CKD among diabetic, hypertensive, and patients>60 years with AUCs of 0.663 (0.625-0.707), 0.678 (0.663-0.691), and 0.681 (0.671-0.691), respectively. In addition, 12-lead and 1-lead ECG-based models detected CKD with similar accuracy among male (AUCs of 0.764 (0.755-0.772) and 0.742 (0.733-0.750), respectively) and female patients (AUCs of 0.756 (0.745-0.768) and 0.735 (0.723-0.747), respectively). Detailed results for 12-lead and 1-lead ECG model performance in the internal test and validation datasets are presented in Table 2 and Table 3. AUC curves for the 12-lead and 1-lead ECG models are illustrated in FIG. 3.

TABLE 2

| | | | | |
|---|---|---|---|---|
| AUC | Sensitivity | Specificity | PPV | NPV |
| (95% CI) | (95% CI) | (95% CI) | (95% CI) | (95% CI) |

12-lead ECG models

| | | | | | |
|---|---|---|---|---|---|
| Any stage CKD | 0.767 (0.76- | 0.699 (0.699- | 0.698 (0.698- | 0.443 (0.443- | 0.871 (0.871- |
| | 0.773) | 0.699) | 0.698) | 0.443) | 0.871) |
| Mild CKD | 0.753 (0.735- | 0.75 (0.75- | 0.644 (0.644- | 0.064 (0.064- | 0.987 (0.987- |
| | 0.77) | 0.75) | 0.644) | 0.064) | 0.987) |
| Moderate- | 0.759 (0.75- | 0.785 (0.785- | 0.598 (0.598- | 0.271 (0.271- | 0.936 (0.936- |
| severe CKD | 0.767) | 0.785) | 0.598) | 0.271) | 0.936) |
| ESRD | 0.783 (0.773- | 0.704 (0.704- | 0.726 (0.726- | 0.237 (0.237- | 0.953 (0.953- |
| | 0.793) | 0.704) | 0.726) | 0.237) | 0.953) |

High Risk Subgroup analyses for any stage CKD

| | | | | | |
|---|---|---|---|---|---|
| Diabetic patients | 0.747 (0.707- | 0.699 (0.699- | 0.682 (0.682- | 0.906 (0.906- | 0.342 (0.342- |
| | 0.783) | 0.699) | 0.682) | 0.906) | 0.342) |
| Hypertensive | 0.714 (0.701- | 0.659 (0.659- | 0.66 (0.66- | 0.798 (0.798- | 0.487 (0.487- |
| patients | 0.726) | 0.659) | 0.66) | 0.798) | 0.487) |
| Age >60 years | 0.706 (0.697- | 0.604 (0.604- | 0.701 (0.701- | 0.397 (0.397- | 0.844 (0.844- |
| | 0.716) | 0.604) | 0.701) | 0.397) | 0.844) |
| Male | 0.764 (0.755- | 0.727 (0.727- | 0.666 (0.666- | 0.485 (0.485- | 0.849 (0.849- |
| | 0.772) | 0.727) | 0.666) | 0.485) | 0.849) |
| Female | 0.756 (0.745- | 0.699 (0.699- | 0.680 (0.680- | 0.351 (0.351- | 0.902 (0.902- |
| | 0.768) | 0.699) | 0.680) | 0.351) | 0.902) |

Screening cohort (age <60)

| | | | | | |
|---|---|---|---|---|---|
| Any stage CKD | 0.843 (0.836- | 0.761 (0.761- | 0.787 (0.787- | 0.57 (0.57- | 0.899 (0.899- |
| | 0.852) | 0.761) | 0.787) | 0.57) | 0.899) |
| Mild CKD | 0.795 (0.766- | 0.702 (0.702- | 0.748 (0.748- | 0.075 (0.075- | 0.989 (0.989- |
| | 0.823) | 0.702) | 0.748) | 0.075) | 0.989) |
| Moderate- | 0.854 (0.842- | 0.792 (0.792- | 0.787 (0.787- | 0.373 (0.373- | 0.959 (0.959- |
| severe CKD | 0.865) | 0.792) | 0.787) | 0.373) | 0.959) |
| ESRD | 0.842 (0.831- | 0.746 (0.746- | 0.791 (0.791- | 0.394 (0.394- | 0.945 (0.945- |
| | 0.853) | 0.746) | 0.791) | 0.394) | 0.945) |

TABLE 3

| | | | | |
|---|---|---|---|---|
| AUC | Sensitivity | Specificity | PPV | NPV |
| (95% CI) | (95% CI) | (95% CI) | (95% CI) | (95% CI) |

1-lead ECG models

| | | | | | |
|---|---|---|---|---|---|
| Any stage CKD | 0.744 (0.737- | 0.723 (0.723- | 0.643 (0.643- | 0.41 (0.41- | 0.871 (0.871- |
| | 0.751) | 0.723) | 0.643) | 0.41) | 0.871) |
| Mild CKD | 0.746 (0.728- | 0.735 (0.735- | 0.66 (0.66- | 0.066 (0.066- | 0.987 (0.987- |
| | 0.764) | 0.735) | 0.66) | 0.066) | 0.987) |
| Moderate- | 0.735 (0.726- | 0.732 (0.732- | 0.618 (0.618- | 0.267 (0.267- | 0.924 (0.924- |
| severe CKD | 0.744) | 0.732) | 0.618) | 0.267) | 0.924) |
| ESRD | 0.757 (0.748- | 0.738 (0.738- | 0.647 (0.647- | 0.202 (0.202- | 0.953 (0.953- |
| | 0.767) | 0.738) | 0.647) | 0.202) | 0.953) |

High Risk Subgroup analyses for any stage CKD

| | | | | | |
|---|---|---|---|---|---|
| Diabetic patients | 0.663 (0.625- | 0.819 (0.819- | 0.457 (0.457- | 0.868 (0.868- | 0.367 (0.367- |
| | 0.707) | 0.819) | 0.457) | 0.868) | 0.367) |
| Hypertensive | 0.678 (0.663- | 0.713 (0.713- | 0.555 (0.555- | 0.765 (0.765- | 0.487 (0.487- |
| patients | 0.691) | 0.713) | 0.555) | 0.765) | 0.487) |
| Age >60 years | 0.681 (0.671- | 0.679 (0.679- | 0.588 (0.588- | 0.35 (0.35- | 0.849 (0.849- |
| | 0.691) | 0.679) | 0.588) | 0.35) | 0.849) |
| Male | 0.742 (0.733- | 0.747 (0.747- | 0.612 (0.612- | 0.455 (0.455- | 0.848 (0.848- |
| | 0.75) | 0.747) | 0.612) | 0.455) | 0.848) |
| Female | 0.735 (0.723- | 0.672 (0.672- | 0.681 (0.681- | 0.342 (0.342- | 0.894 (0.894- |
| | 0.747) | 0.672) | 0.681) | 0.342) | 0.894) |

Screening cohort (age <60)

| | | | | | |
|---|---|---|---|---|---|
| Any stage CKD | 0.824 (0.815- | 0.812 (0.812- | 0.705 (0.705- | 0.506 (0.506- | 0.91 (0.91- |
| | 0.832) | 0.812) | 0.705) | 0.506) | 0.91) |
| Mild CKD | 0.819 (0.791- | 0.771 (0.771- | 0.758 (0.758- | 0.085 (0.085- | 0.991 (0.991- |
| | 0.844) | 0.771) | 0.758) | 0.085) | 0.991) |
| Moderate- | 0.828 (0.816- | 0.814 (0.814- | 0.713 (0.713- | 0.313 (0.313- | 0.96 (0.96- |
| severe CKD | 0.84) | 0.814) | 0.713) | 0.313) | 0.96) |
| ESRD | 0.82 (0.808- | 0.82 (0.82- | 0.693 (0.693- | 0.328 (0.328- | 0.955 (0.955- |
| | 0.831) | 0.82) | 0.693) | 0.328) | 0.955) |

The model performed similarly in detecting CKD in subset populations of patients with albuminuria, patients with corresponding laboratory testing and documented eGFR, and in both ambulatory and in-hospital patients. In patients with both a CKD diagnosis and eGFR estimated to be less than 60 mL/min, the AUC was 0.754 (0.737-0.771), and this performance was similar in patients with hyperkalemia with an AUC of 0.741 (0.698-0.787) and without hyperkalemia with an AUC of 0.758 (0.747-0.777). The model also performed well in patients with known albuminuria, with an AUC of 0.734 (0.723-0.745) and had similar performance regardless of the positive to negative ratio in the training set.

To understand the key features of relevance for the deep learning model to be able to detect CKD, two sets of experiments were performed to evaluate the ECG parameters that are important for identifying CKD. The values of these parameters are shown in Table 4 below. Overall values are shown, as well as values for patients with no CKD, mild CKD, moderate CDK, and end-stage renal disease. Statistically significant differences were found in all available ECG variables (heart rate, PR interval, P wave duration, QRS duration, QTc interval, P-wave axis, R-wave axis, T-wave axis) between CKD stages. Most prominently, patients with CKD had prolonged PR interval, prolonged QRS duration, prolonged QTc interval, and skewed T wave axis in comparison to those without CKD.

TABLE 4

| Electro- | | | | Moder- | | |
|---|---|---|---|---|---|---|
| cardiographic | | No | Mild | ate | | P |
| characteristic | Overall | CKD | CKD | CKD | ESRD | value |
| Heart rate, bpm | 81.1 ± | 80.5 ± | 85.7 ± | 82.8 ± | 80.9 ± | <0.001 |
| | 21.8 | 22.1 | 21.2 | 21.3 | 19.2 | |
| PR interval, ms | 166.0 ± | 164.5 ± | 165.8 ± | 172.5 ± | 168.4 ± | <0.001 |
| | 37.7 | 36.9 | 36.8 | 43.2 | 34.6 | |
| P wave | 52.2 ± | 52.1 ± | 52.1 ± | 51.9 ± | 53.6 ± | <0.001 |
| duration, ms | 9.6 | 9.2 | 11.2 | 11.1 | 9.2 | |
| QRS duration, | 101.0 ± | 98.7 ± | 112.6 ± | 111.8 ± | 98.9 ± | <0.001 |
| ms | 29.0 | 27.2 | 36.3 | 35.6 | 24.7 | |
| QTc interval, | 457.6 ± | 452.6 ± | 477.4 ± | 474.5 ± | 468.3 ± | <0.001 |
| ms | 66.7 | 70.3 | 60.2 | 55.2 | 43.7 | |
| P-wave axis | 50.2 ± | 50.3 ± | 50.6 ± | 50.2 ± | 48.9 ± | <0.001 |
| | 27.5 | 27.0 | 26.6 | 29.9 | 26.2 | |
| R-wave axis | 23.6 ± | 23.2 ± | 38.0 ± | 24.6 ± | 22.1 ± | <0.001 |
| | 60.2 | 51.1 | 85.1 | 75.1 | 57.2 | |
| T-wave axis | 57.5 ± | 54.1 ± | 61.1 ± | 67.6 ± | 67.0 ± | <0.001 |
| | 57.6 | 54.9 | 64.4 | 66.3 | 57.3 | |

Secondly, LIME was used to identify which ECG segments were particularly used in the identification of CKD. FIG. 4 shows examples of LIME-highlighted ECG segments in 12-lead and 1-lead ECG waveforms taken from correctly recognized CKD and healthy control patients in the held-out test set. In both examples, the LIME-highlighted ECG features focused mostly on QRS complexes and PR intervals. In addition, QRS complexes and PR intervals in limb leads were most frequently highlighted, potentially denoting CKD-associated electrophysiological alterations.

The external validation cohort consisted of a total of 896,620 ECGs among 312,145 patients. The prevalence of mild CKD was 1.2% while 3.6% had moderate-severe CKD, and 0.9% had ESRD. The mean age of the external validation cohort was 56.7±18.7 years and 50.4% were female. The proportion of Caucasians was 47.5%, while 3.6% were black, 12.3% were Asians, and 36.6% had other or unknown race. Demographic and clinical characteristics are shown above in Table 1.

In the external validation dataset, the performance of the 12-lead ECG model and the 1-lead ECG models was comparable to the primary cohort. The 12-lead ECG-based model achieved an AUC of 0.709 (0.708-0.710) in discriminating any stage CKD, with a sensitivity of 0.212 (0.210-0.214) and specificity of 0.926 (0.926-0.927). Additionally, 12-lead ECG-based model achieved an AUC of 0.679 (0.675-0.682) in discriminating mild CKD, AUC of 0.714 (0.713-0.716) in discriminating moderate-severe CKD, and AUC of 0.767 (0.764-0.769) in discriminating ESRD. 1-lead ECG-based model detected any stage CKD with an AUC of 0.701 (0.700-0.702), mild stage CKD with an AUC of 0.671 (0.668-0.674), moderate-severe CKD with an AUC of 0.694 (0.692-0.695), and ESRD with an AUC of 0.780 (0.778-0.782).

Consistent with the primary cohort in which the model achieved higher CKD detection accuracy among younger patients, 12-lead and 1-lead ECG-based models achieved AUCs of 0.784 (0.782-0.786) and 0.777 (0.775-0.779) in detecting any stage CKD among subjects under 60 years of age, respectively. However, the 12-lead ECG-based model's CKD discrimination accuracy was somewhat lower in high-risk subgroups with an AUC of 0.699 (0.697-0.702) among diabetic, AUC of 0.712 (0.710-0.714) among hypertensive, and AUC of 0.660 (0.658-0.661) among subjects over 60 years of age. Detailed results for 12-lead and 1-lead ECG-based model performance in the external validation cohort are presented in Table 5 and Table 6.

TABLE 5

| | | | | |
|---|---|---|---|---|
| AUC | Sensitivity | Specificity | PPV | NPV |
| (95% CI) | (95% CI) | (95% CI) | (95% CI) | (95% CI) |

12-lead ECG models

| | | | | | |
|---|---|---|---|---|---|
| Any stage CKD | 0.709 (0.708- | 0.573 (0.571- | 0.720 (0.719- | 0.276 (0.275- | 0.900 (0.900- |
| | 0.710) | 0.575) | 0.721) | 0.278) | 0.901) |
| Mild CKD | 0.679 (0.675- | 0.525 (0.519- | 0.720 (0.719- | 0.047 (0.046- | 0.983 (0.983- |
| | 0.682) | 0.530) | 0.721) | 0.048) | 0.983) |
| Moderate- | 0.714 (0.713- | 0.585 (0.582- | 0.720 (0.719- | 0.141 (0.140- | 0.956 (0.956- |
| severe CKD | 0.716) | 0.588) | 0.720) | 0.143) | 0.957) |
| ESRD | 0.767 (0.764- | 0.665 (0.660- | 0.720 (0.719- | 0.083 (0.082- | 0.983 (0.982- |
| | 0.769) | 0.670) | 0.721) | 0.084) | 0.983) |

High Risk Subgroup analyses for any stage CKD

| | | | | | |
|---|---|---|---|---|---|
| Diabetic patients | 0.699 (0.697- | 0.620 (0.615- | 0.670 (0.665- | 0.711 (0.708- | 0.573 (0.568- |
| | 0.702) | 0.625) | 0.674) | 0.716) | 0.577) |
| Hypertensive | 0.712 (0.710- | 0.555 (0.552- | 0.741 (0.740- | 0.378 (0.375- | 0.855 (0.854- |
| patients | 0.714) | 0.558) | 0.742) | 0.380) | 0.856) |
| Age >60 years | 0.660 (0.658- | 0.582 (0.579- | 0.648 (0.647- | 0.349 (0.347- | 0.827 (0.826- |
| | 0.661) | 0.584) | 0.649) | 0.350) | 0.828) |
| Male | 0.719 (0.718- | 0.593 (0.590- | 0.721 (0.720- | 0.387 (0.384- | 0.857 (0.856- |
| | 0.721) | 0.596) | 0.723) | 0.389) | 0.858) |
| Female | 0.728 (0.726- | 0.537 (0.533- | 0.769 (0.768- | 0.279 (0.276- | 0.909 (0.908- |
| | 0.730) | 0.541) | 0.770) | 0.281) | 0.910) |

Screening cohort (age <60)

| | | | | | |
|---|---|---|---|---|---|
| Any stage CKD | 0.784 (0.782- | 0.552 (0.548- | 0.843 (0.843- | 0.326 (0.323- | 0.932 (0.931- |
| | 0.786) | 0.556) | 0.845) | 0.328) | 0.933) |
| Mild CKD | 0.763 (0.757- | 0.524 (0.516- | 0.843 (0.843- | 0.063 (0.061- | 0.989 (0.989- |
| | 0.767) | 0.536) | 0.844) | 0.065) | 0.989) |
| Moderate- | 0.780 (0.777- | 0.548 (0.543- | 0.843 (0.843- | 0.128 (0.126- | 0.978 (0.978- |
| severe CKD | 0.782) | 0.556) | 0.844) | 0.130) | 0.979) |
| ESRD | 0.841 (0.839- | 0.649 (0.644- | 0.843 (0.843- | 0.155 (0.153- | 0.982 (0.982- |
| | 0.844) | 0.656) | 0.845) | 0.158) | 0.982) |

TABLE 6

| | | | | |
|---|---|---|---|---|
| AUC | Sensitivity | Specificity | PPV | NPV |
| (95% CI) | (95% CI) | (95% CI) | (95% CI) | (95% CI) |

1-lead ECG models

| | | | | | |
|---|---|---|---|---|---|
| Any stage CKD | 0.701 (0.700- | 0.660 (0.658- | 0.635 (0.634- | 0.252 (0.251- | 0.909 (0.908- |
| | 0.702) | 0.662) | 0.635) | 0.253) | 0.910) |
| Mild CKD | 0.671 (0.668- | 0.617 (0.612- | 0.635 (0.634- | 0.043 (0.042- | 0.984 (0.984- |
| | 0.674) | 0.621) | 0.636) | 0.043) | 0.985) |
| Moderate- | 0.694 (0.692- | 0.649 (0.645- | 0.635 (0.634- | 0.123 (0.122- | 0.958 (0.958- |
| severe CKD | 0.695) | 0.652) | 0.636) | 0.124) | 0.959) |
| ESRD | 0.780 (0.778- | 0.784 (0.780- | 0.635 (0.634- | 0.075 (0.075- | 0.987 (0.987- |
| | 0.782) | 0.788) | 0.636) | 0.076) | 0.987) |

High Risk Subgroup analyses for any stage CKD

| | | | | | |
|---|---|---|---|---|---|
| Diabetic patients | 0.678 (0.674- | 0.710 (0.706- | 0.544 (0.539- | 0.671 (0.667- | 0.588 (0.584- |
| | 0.681) | 0.713) | 0.549) | 0.674) | 0.593) |
| Hypertensive | 0.697 (0.696- | 0.627 (0.623- | 0.661 (0.659- | 0.343 (0.340- | 0.862 (0.861- |
| patients | 0.699) | 0.630) | 0.662) | 0.346) | 0.863) |
| Age >60 years | 0.657 (0.655- | 0.646 (0.643- | 0.583 (0.582- | 0.335 (0.332- | 0.836 (0.834- |
| | 0.658) | 0.649) | 0.585) | 0.336) | 0.837) |
| Male | 0.722 (0.720- | 0.667 (0.665- | 0.661 (0.659- | 0.368 (0.366- | 0.870 (0.869- |
| | 0.723) | 0.669) | 0.662) | 0.370) | 0.871) |
| Female | 0.706 (0.704- | 0.648 (0.644- | 0.650 (0.648- | 0.236 (0.234- | 0.917 (0.916- |
| | 0.708) | 0.652) | 0.651) | 0.237) | 0.918) |

Screening cohort (age <60)

| | | | | | |
|---|---|---|---|---|---|
| Any stage CKD | 0.777 (0.775- | 0.694 (0.690- | 0.728 (0.726- | 0.259 (0.257- | 0.946 (0.945- |
| | 0.779) | 0.697) | 0.729) | 0.261) | 0.946) |
| Mild CKD | 0.752 (0.748- | 0.657 (0.647- | 0.728 (0.726- | 0.046 (0.045- | 0.991 (0.990- |
| | 0.757) | 0.666) | 0.729) | 0.047) | 0.991) |
| Moderate- | 0.764 (0.761- | 0.674 (0.668- | 0.728 (0.726- | 0.094 (0.092- | 0.982 (0.981- |
| severe CKD | 0.768) | 0.679) | 0.729) | 0.095) | 0.982) |
| ESRD | 0.837 (0.834- | 0.799 (0.794- | 0.728 (0.727- | 0.115 (0.114- | 0.988 (0.988- |
| | 0.839) | 0.804) | 0.729) | 0.117) | 0.988 |

The 12-lead ECG-based model had good accuracy in identifying any stage CKD and higher accuracy in detecting CKD in patients under 60 years of age. Accuracy also improved along with the worsening CKD stage. These results were validated in a separate health care system, that also showed good discrimination accuracy for the presence of any stage CKD in the whole study population and higher discrimination accuracy among patients under 60 years of age. While 12-lead ECGs are widely available in the healthcare unit settings, rapid adoption of wearable technology has also introduced opportunities for large-scale data collection outside of formal healthcare settings. The 1-lead ECG-based model showed good discrimination accuracy for CKD in young patients, suggesting artificial intelligence may possess significant potential in widescale screening in this patient population. One-lead ECGs could also increase screening rates in high-risk patients, as illustrated in FIGS. 12 and 13.

Low awareness of CKD and limitations in current screening measures highlight the urgency of novel screening strategies to increase detection rates of early-stage CKD. Being non-invasive and often obtained in the clinic, ECGs are often the first line of clinical evaluation. In the current healthcare system, 74% of ECGs obtained did not have laboratory testing of kidney function within 30 days. Previous studies have demonstrated that the cost-effectiveness of CKD screening is highly dependent on patient risk factor profile and CKD probability, and there has been debate on whether CKD screening should be targeted only to high-risk patients, or also extend to patients without risk factors for CKD). Although screening high-risk patients is guideline-recommended, testing rates remain low as only about 20% of high-risk patients receive guideline-recommended assessment in the U.S. Consequently, most of the high-risk patients are likely to be unaware of underlying CKD. Moreover, a substantial proportion of all CKD patients are not high-risk patients and hence not recommended to be screened regularly, which further highlights the need for novel screening methods

The model performed better at detecting CKD in younger patients, whereas detection accuracy was lower in older and high-risk patients. Reasons for this observation are not fully clear but may be due to the fact that younger patients in general have fewer comorbidities, meaning that any detected ECG abnormalities may be especially meaningful and specific. Although older age is a well-known risk marker for CKD, the prevalence of CKD in younger patients is also notably high in the U.S. (8-10% in <65 years). Remarkably, however, awareness of underlying CKD is also very low in younger patients, as only about 8% are aware of the disease. Given the availability of effective low-risk CKD treatments and the reversibility of CKD, there are substantial potential benefits for detecting and treating CKD, especially in the young. A recent paper by Kwon et al also used data from ECG waveforms in addition to age and sex to develop a model to detect changes in eGFR, which can include both patients with acute kidney injury (e.g., dehydration, pharmacotherapy, urinary tract obstruction) as well as chronic kidney disease. Their model achieved a slightly higher performance with an AUC of 0.86-0.91, however reaffirms the overall conclusion that renal abnormalities can be detected by CKD within large cohorts across multiple international sites

The ECG-based deep learning model was able to detect CKD with good discrimination accuracy in multiple study populations and with particularly high accuracy in patients under 60 years of age. These results suggest that deep learning-based ECG analysis may provide additional value in detecting various CKD stages, especially in younger patients. The clinical significance of this study lies in the importance of novel screening methods for early detection of CKD, which is crucial to enable early treatment and prevent disease progression.

FIG. 7 illustrates a block diagram of system 300 that can be used to analyze kidney health in an individual based on ECG data. In general, the system 300 can be used to train and/or implement any of the models disclosed herein. The system 300 can include one or more processing devices 310, which can each include any one or more of a processor 312, a memory 314, a display 316, a user input device 318, and/or other components. The memory 314 can include machine-readable instructions for executing the models and methods disclosed herein, and/or other models and methods. The processor 312 can execute these instructions to implement these models and methods. The memory 314 can also store data associated with the methods, such as 12-lead ECG data and 1-lead ECG data.

The processing device 310 can include any suitable processing device, such as general purpose computer systems, microprocessors, digital signal processors, micro-controllers, application specific integrated circuits (ASICs), programmable logic devices (PLDs) field programmable logic devices (FPLDs), programmable gate arrays (PGAs), field programmable gate arrays (FPGAs), mobile devices such as mobile telephones, personal digital assistants (PDAs), or tablet computers, local servers, remote servers, wearable computers, or the like. The memory device 114 can include any suitable memory device and/or machine-readable medium that is capable of storing, encoding, and/or carrying a set of instructions for execution by a processing device and that cause the processing device to perform and/or implement any of the features discussed herein, including solid-state memories, optical media, magnetic media, random access memory (RAM), read only memory (ROM), a floppy disk, a hard disk, a CD ROM, a DVD ROM, flash memory, or other computer readable medium that is read from and/or written to by a magnetic, optical, or other reading and/or writing system that is coupled to the processing device, can be used for the memory or memories.

The display 316 can be used to display any information associated with the features disclosed herein, including the results of the classification analysis by the machine learning model. The display device 316 can be any known display technology, including but not limited to display devices using Liquid Crystal Display (LCD) or Light Emitting Diode (LED) technology. The user input device 318 can be used to allow the user to interact with the system 300 for any suitable purpose, including initiating, pausing, or terminating the analysis by the machine learning model; adjusting any parameters of the analysis, etc. In some implementations, the system 300 includes an ECG system 320 that generates the ECG data that is used in the models and methods disclosed herein. The ECG system 320 can generally be any suitable type of system or device configured to obtain ECG data. In other implementations, the system 300 does not include the ECG system 320, but instead receives ECG data from an external source.

FIG. 8 shows a flow chart of a method 400 for analyzing the health of a kidney of an individual. Step 410 of method 400 includes receiving data associated with one or more cardiac characteristics of the individual. This data can include 1-lead ECG data, 12-lead ECG data, and/or any other combination of data. The cardiac characteristics can include a heart rate, a PR interval, a P wave duration, a QRS duration, a QTc interval, a P-wave axis, an R-wave axis, a T-wave axis, other characteristics, or any combination thereof. In some implementations, the data includes ECG data associated with the QRS complex of a plurality of heartbeats of the individual, with the PR interval of the plurality of heartbeats of the individual, or both.

Step 420 of method 400 includes inputting the data into a machine learning model. Any suitable machine learning model can be used. In some implementations, the model is a convolutional neural network that analyzes ECG data starting with atrous convolutions followed by subsequent multi-channel ID convolutions. After the initial atrous layers, the model incorporates convolutional layers with an inverted residual structure where the input and output are bottleneck 9 layers with an intermediate expansion layer. In each set of expansion layers with bottleneck layers preceding and succeeding, the number of input channels gradually increased to allow for integration of information across ECG leads.

The model can be trained as disclosed herein. For example, the model can be a 1-lead or 12-lead ECG model where the training data includes ECG for a plurality of patients, and an indication of whether or not each respective patient was diagnosed with CKD. For patients with a diagnosis of CKD, the ECG data originated from a time not more than 1 year prior to the CKD diagnosis. For patients with no diagnosis of CKD, the ECG data can generally originate from any point in time in their health record. The model can be trained with this training data to correlate ECG data with a CKD diagnosis. In some implementations, the model is initialized with random weights and trained using a binary cross entropy loss function for up to 100 epochs, with an ADAM optimizer and an initial learning rate of 1e-4. Local Interpretable Model-agnostic Explanation (LIME) operations were performed to identify relevant features in the ECG data by iteratively randomly perturbing 0.5% of the ECG data and identifying which changes most impacted model performance.

Step 430 of method 400 includes receiving an output from the machine learning model indicative of the kidney health of the individual. In some implementations, the output of the machine learning model includes an indication of a presence of chronic kidney disease (CKD) in the individual or an indication of an absence of CKD in the individual. The indication could include a specific yes/no as to the presence of CKD, and/or could include a percentage chance that the individual has CKD. In some implementations, the indication of the presence of CKD in the individual includes an indication of a CKD stage of the individual. In some implementations, the CKD stage of the individual includes mild CKD, moderate-severe CKD, or end-stage renal disease (ESRD). The indication could include a specific identification of which CKD stage the individual is in, and/or could include a percentage chance that the individual is in each CKD stage (or at least two CKD stages).

In some implementations, the data includes 12-lead ECG. In some of these implementations, the model identifies mild CKD with an AUC value of between 0.73 and 0.78. In some of these implementations, the model identifies moderate-severe CKD with an AUC value of between 0.75 and 0.77. In some of these implementations, the model identifies ESRD with an AUC value of between 0.73 and 0.76. In some of these implementations, the model identifies the presence of CKD with an AUC value of between 0.76 and 0.77. In some of these implementations, the model identifies the presence of CKD with a sensitivity of between 0.6 and 0.7, a specificity of between 0.6 and 0.7, or both.

In some implementations, the data includes 1-lead ECG. In some of these implementations, the model identifies mild CKD with an AUC value of between 0.72 and 0.77. In some of these implementations, the model identifies moderate-severe CKD with an AUC value of between 0.72 and 0.75. In some of these implementations, the model identifies ESRD with an AUC value of between 0.74 and 0.77. In some of these implementations, the model identifies the presence of CKD with an AUC value of between 0.73 and 0.76. In some of these implementations, the model identifies the presence of CKD with a sensitivity of between 0.7 and 0.8, a specificity of between 0.6 and 0.7, or both.

### Alternative Implementations

Alternative Implementation 1. A method for analyzing kidney health in an individual, the method comprising: receiving data associated with one or more cardiac characteristics of the individual; inputting the data associated with the one or more cardiac characteristics of the individual into a machine learning model; and receiving an output from the machine learning model indicative of the kidney health of the individual.

Alternative Implementation 2. The method of Alternative Implementation 1, wherein the output of the machine learning model includes an indication of a presence of chronic kidney disease (CKD) in the individual or an indication of an absence of CKD in the individual.

Alternative Implementation 3. The method of Alternative Implementation 2, wherein the indication of the presence of CKD in the individual includes an indication of a CKD stage of the individual.

Alternative Implementation 4. The method of Alternative Implementation 3, wherein the CKD stage of the individual includes mild CKD, moderate-severe CKD, or end-stage renal disease (ESRD)

Alternative Implementation 5. The method of any one of Alternative Implementations 2 to 4, wherein the data associated with the one or more cardiac characteristics of the individual includes 12-lead electrocardiograph (ECG) data.

Alternative Implementation 6. The method of Alternative Implementation 5, wherein the machine learning model is trained to identify mild CKD in the individual with an area under a receiver operating characteristic curve (AUC) value of between 0.735 and 0.770.

Alternative Implementation 7. The method of Alternative Implementation 5 or Alternative Implementation 6, wherein the machine learning model is trained to identify moderate-severe CKD in the individual with an AUC value of between 0.750 and 0.767.

Alternative Implementation 8. The method of any one of Alternative Implementations 5 to 7, wherein the machine learning model is trained to identify ESRD in the individual with an AUC value of between 0.730 and 0.752.

Alternative Implementation 9. The method of any one of Alternative Implementations 5 to 8, wherein the machine learning model is trained to identify the presence of CKD in the individual with an AUC value of between 0.760 and 0.773.

Alternative Implementation 10. The method of any one of Alternative Implementations 5 to 9, wherein the machine learning model is trained to identify the presence of CKD in the individual with a sensitivity of 0.699.

Alternative Implementation 11. The method of any one of Alternative Implementations 5 to 10, wherein the machine learning model is trained to identify the presence of CKD in the individual with a specificity of 0.698.

Alternative Implementation 12. The method of any one of Alternative Implementations 5 to 11, wherein the individual is less than 60 years old, and wherein the machine learning model is trained to identify the presence of CKD in the individual with an AUC value of between 0.834 and 0.852.

Alternative Implementation 13. The method of any one of Alternative Implementations 5 to 12, wherein the individual is less than 60 years old, and wherein the machine learning model is trained to identify the presence of CKD in the individual with a sensitivity of 0.761.

Alternative Implementation 14. The method of any one of Alternative Implementations 5 to 13, wherein the individual is less than 60 years old, and wherein the machine learning model is trained to identify the presence of CKD in the individual with a specificity of 0.787.

Alternative Implementation 15. The method of any one of Alternative Implementations 5 to 14, wherein the individual has diabetes, and wherein the machine learning model is trained to identify the presence of CKD in the individual with an AUC value of between 0.707 and 0.783.

Alternative Implementation 16. The method of any one of Alternative Implementations 5 to 15, wherein the individual has hypertension, and wherein the machine learning model is trained to identify the presence of CKD in the individual with an AUC value of between 0.701 and 0.726.

Alternative Implementation 17. The method of any one of Alternative Implementations 5 to 16, wherein the individual is greater than 60 years old, and wherein the machine learning model is trained to identify the presence of CKD in the individual with an AUC value of between 0.697 and 0.716.

Alternative Implementation 18. The method of any one of Alternative Implementations 5 to 17, wherein the individual is male, and wherein the machine learning model is trained to identify the presence of CKD in the individual with an AUC value of between 0.755 and 0.772.

Alternative Implementation 19. The method of any one of Alternative Implementations 5 to 18, wherein the individual is female, and wherein the machine learning model is trained to identify the presence of CKD in the individual with an AUC value of between 0.745 and 0.768.

Alternative Implementation 20. The method of any one of Alternative Implementations 2 to 4, wherein the data associated with the one or more cardiac characteristics of the individual includes 1-lead electrocardiograph (ECG) data.

Alternative Implementation 21. The method of Alternative Implementation 20, wherein the machine learning model is trained to identify mild CKD in the individual with an area under a receiver operating characteristic curve (AUC) value of between 0.728 and 0.764.

Alternative Implementation 22. The method of Alternative Implementation 20 or Alternative Implementation 21, wherein the machine learning model is trained to identify moderate-severe CKD in the individual with an AUC value of between 0.726 and 0.744.

Alternative Implementation 23. The method of any one of Alternative Implementations 20 to 22, wherein the machine learning model is trained to identify ESRD in the individual with an AUC value of between 0.748 and 0.767.

Alternative Implementation 24. The method of any one of Alternative Implementations 20 to 23, wherein the machine learning model is trained to identify the presence of CKD in the individual with an AUC value of between 0.737 and 0.751.

Alternative Implementation 25. The method of any one of Alternative Implementations 20 to 24, wherein the machine learning model is trained to identify the presence of CKD in the individual with a sensitivity of 0.723.

Alternative Implementation 26. The method of any one of Alternative Implementations 20 to 25, wherein the machine learning model is trained to identify the presence of CKD in the individual with a specificity of 0.643.

Alternative Implementation 27. The method of any one of Alternative Implementations 20 to 26, wherein the individual is less than 60 years old, and wherein the machine learning model is trained to identify the presence of CKD in the individual with an AUC value of between 0.814 and 0.833.

Alternative Implementation 28. The method of any one of Alternative Implementations 20 to 27, wherein the individual is less than 60 years old, and wherein the machine learning model is trained to identify the presence of CKD in the individual with a sensitivity of 0.812.

Alternative Implementation 29. The method of any one of Alternative Implementations 20 to 28, wherein the individual is less than 60 years old, and wherein the machine learning model is trained to identify the presence of CKD in the individual with a specificity of 0.705.

Alternative Implementation 30. The method of any one of Alternative Implementations 20 to 29, wherein the individual has diabetes, and wherein the machine learning model is trained to identify the presence of CKD in the individual with an AUC value of between 0.625 and 0.707.

Alternative Implementation 31. The method of any one of Alternative Implementations 20 to 30, wherein the individual has hypertension, and wherein the machine learning model is trained to identify the presence of CKD in the individual with an AUC value of between 0.663 and 0.691.

Alternative Implementation 32. The method of any one of Alternative Implementations 20 to 31, wherein the individual is greater than 60 years old, and wherein the machine learning model is trained to identify the presence of CKD in the individual with an AUC value of between 0.671 and 0.691.

Alternative Implementation 33. The method of any one of Alternative Implementations 20 to 32, wherein the individual is male, and wherein the machine learning model is trained to identify the presence of CKD in the individual with an AUC value of between 0.733 and 0.750.

Alternative Implementation 34. The method of any one of Alternative Implementations 20 to 33, wherein the individual is female, and wherein the machine learning model is trained to identify the presence of CKD in the individual with an AUC value of between 0.723 and 0.747.

Alternative Implementation 35. The method of any one of Alternative Implementations 1 to 34, wherein the one or more cardiac characteristics of the individual includes a heart rate, a PR interval, a P wave duration, a QRS duration, a QTc interval, a P-wave axis, an R-wave axis, a T-wave axis, or any combination thereof.

Alternative Implementation 36. The method of any one of Alternative Implementations 1 to 35, wherein the machine learning model is trained to identify a presence of CKD in the individual in response to the data indicating the individual has a prolonged PR interval, a prolonged QRS duration, a prolonged QTc interval, a skewed T-wave, or any combination thereof.

Alternative Implementation 37. The method of any one of Alternative Implementations 1 to 36, wherein the data associated with the one or more cardiac characteristics of the individual includes ECG data associated with one or more QRS complexes of a heartbeat of the individual, one or more PR intervals of the heartbeat of the individual, or both.

Alternative Implementation 38. The method of any one of Alternative Implementations 1 to 37, wherein the machine learning model includes a convolutional neural network trained to identify a presence of CKD in the individual, to determine a stage of the CKD in the individual, or both.

Alternative Implementation 39. A system for analyzing kidney health in an individual, the system comprising: at least one memory device configured to receive and store data associated with the one or more cardiac characteristics of the individual; and at least one processing device configured to implement a machine learning model, the machine learning model being configured to receive the data associated with the one or more cardiac characteristics of the individual and output an indication of the kidney health of the individual.

Alternative Implementation 40. The system of Alternative Implementation 39, wherein the at least one processing device is configured to implement the method of any one of Alternative Implementations 2 to 38.

One or more elements or aspects or steps, or any portion(s) thereof, from one or more of any of the Alternative Implementations or claims below can be combined with one or more elements or aspects or steps, or any portion(s) thereof, from one or more of any of the other Alternative Implementations or claims or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A method for analyzing kidney health in an individual, the method comprising:
receiving data associated with one or more cardiac characteristics of the individual;
inputting the data associated with the one or more cardiac characteristics of the individual into a machine learning model; and
receiving an output from the machine learning model indicative of the kidney health of the individual.

2. The method of claim 1, wherein the output of the machine learning model includes an indication of a presence of chronic kidney disease (CKD) in the individual or an indication of an absence of CKD in the individual.

3. The method of claim 2, wherein the indication of the presence of CKD in the individual includes an indication of a CKD stage of the individual.

4. The method of claim 3, wherein the CKD stage of the individual includes mild CKD, moderate-severe CKD, or end-stage renal disease (ESRD).

5. The method of claim 2, wherein the data associated with the one or more cardiac characteristics of the individual includes 12-lead electrocardiograph (ECG) data.

6. The method of claim 5, wherein the machine learning model is trained to identify mild CKD in the individual with an area under a receiver operating characteristic curve (AUC) value of between 0.73 and 0.77.

7. The method of claim 5, wherein the machine learning model is trained to identify moderate-severe CKD in the individual with an AUC value of between 0.75 and 0.77.

8. The method of claim 5, wherein the machine learning model is trained to identify ESRD in the individual with an AUC value of between 0.73 and 0.76.

9. The method of claim 5, wherein the machine learning model is trained to identify the presence of CKD in the individual with an AUC value of between 0.76 and 0.78.

10. The method of claim 5, wherein the machine learning model is trained to identify the presence of CKD in the individual with a sensitivity of between 0.6 and 0.7, a specific of between 0.6 and 0.7, or both.

11. The method of claim 2, wherein the data associated with the one or more cardiac characteristics of the individual includes 1-lead electrocardiograph (ECG) data.

12. The method of claim 11, wherein the machine learning model is trained to identify mild CKD in the individual with an area under a receiver operating characteristic curve (AUC) value of between 0.72 and 0.77.

13. The method of claim 11, wherein the machine learning model is trained to identify moderate-severe CKD in the individual with an AUC value of between 0.72 and 0.75.

14. The method of claim 11, wherein the machine learning model is trained to identify ESRD in the individual with an AUC value of between 0.74 and 0.77.

15. The method of claim 11, wherein the machine learning model is trained to identify the presence of CKD in the individual with an AUC value of between 0.73 and 0.76.

16. The method of claim 11, wherein the machine learning model is trained to identify the presence of CKD in the individual with a sensitivity of between 0.7 and 0.8, a specificity of between 0.6 and 0.7, or both.

17. The method of claim 1, wherein the one or more cardiac characteristics of the individual includes a heart rate, a PR interval, a P wave duration, a QRS duration, a QTc interval, a P-wave axis, an R-wave axis, a T-wave axis, or any combination thereof.

18. The method of claim 1, wherein the machine learning model is trained to identify a presence of CKD in the individual in response to the data indicating the individual has a prolonged PR interval, a prolonged QRS duration, a prolonged QTc interval, a skewed T-wave, or any combination thereof.

19. The method of claim 1, wherein the data associated with the one or more cardiac characteristics of the individual includes ECG data associated with one or more QRS complexes of a heartbeat of the individual, one or more PR intervals of the heartbeat of the individual, or both.

20. The method of claim 1, wherein the machine learning model includes a convolutional neural network trained to identify a presence of CKD in the individual, to determine a stage of the CKD in the individual, or both.

21. A system for analyzing kidney health in an individual, the system comprising:
at least one memory device configured to receive and store data associated with the one or more cardiac characteristics of the individual; and
at least one processing device configured to implement a machine learning model, the machine learning model being configured to receive the data associated with the one or more cardiac characteristics of the individual and output an indication of the kidney health of the individual.
